(19) United States
(12) Patent Application Publication
(10) Pub. No.: US 2004/0221376 A1
(43) Pub. Date: Nov. 11, 2004

(54) Swimming goggles

(76) Inventors: Haruo Kawashima, Tokyo (JP); Shunji Fukasawa, Tokyo (JP); Isamu Yamazaki, Tokyo (JP)

Correspondence Address: CLARK & BRODY, 1750 K STREET NW, SUITE 600, WASHINGTON, DC, 20006, US

(21) Appl. No.: 10/787,163

(22) Filed: Feb. 27, 2004

(30) Foreign Application Priority Data
Mar. 7, 2003 (JP) 2003-61913

(51) Int. Cl. A61F 9/02

(52) U.S. Cl. 2/428

(57) ABSTRACT

Swimming goggles improved so that a visual line of a swimming goggles wearer may be easily directed forward during competitive swimming. Each lens of swimming goggles is divided into an intermediate section extending across the lens at a generally middle level as viewed in a vertical direction, an upper section lying above the intermediate section and a lower section lying below the intermediate section. The intermediate section presents a see-through clarity lower than those presented by the upper section and the lower section.

### BACKGROUND OF THE INVENTION

The present invention relates to swimming goggles.

Japanese Utility Model Publication No. 1979-33280A discloses swimming goggles having lenses each comprising an upper section obliquely extending upward so as to get nearer to the wearer's face and a lower section obliquely extending downward so as to get nearer to the wearer's face, both sections obliquely extend at an angle in a range of 10 to 25° relative to the wearer's face. These known swimming goggles achieve a vertical visual field angle a which is larger than the vertical visual field angle achieved by usual flat lenses. According to the disclosure in the foregoing Publication, these known swimming goggles certainly ensures the relatively wide visual field both for upward glance and for downward glance and convenient for competitive swimmers who should look forward with upward glance at an angle of 45° with respect to water surface.

Certainly, with the swimming goggles described in the foregoing Publication, the vertically wide visual field facilitates the swimmer to look forward through the lenses with upward glance at an angle 45° during swimming. However, it is likely that various sights unnecessary for competitive swimming might get from such relatively wide visual field into the swimmers eyes and might impair the swimmers concentration. This is undesirable for the swimmers in competitive swimming.

### SUMMARY OF THE INVENTION

In view of the problem as has been described above, it is an object of the present invention to provide swimming goggles improved so that a visual line of a swimming goggles wearer may be easily directed forward during competitive swimming.

According the present invention, there is provided swimming goggles comprising a pair of lens assemblies, a link connecting said lens assemblies to each other and a strap put around a head of a swimming goggles wearer.

The lens assemblies have a transverse direction horizontally extending across the head and a longitudinal direction vertically extending orthogonally to the transverse direction and the lens included in each of the lens assemblies is divided into an intermediate section extending across the lens at a generally middle level as viewed in a vertical direction, an upper section lying above the intermediate section and a lower section lying below the intermediate section wherein the intermediate section presents a see-through clarity lower than those presented by the upper section and the lower section.

The present invention includes the following embodiments.

The see-through clarity is specified by total luminous transmittance or parallel luminous transmittance of the intermediate sections.

The total luminous transmittance or the parallel luminous transmittance of the intermediate sections is substantially 0%.

Each of the lenses has an inner surface opposed to a swimming goggles wearer's face and an outer surface facing away from the wearer's face and wherein each of the upper section obliquely extends upward so as to get nearer to the face and each of the lower section obliquely extends downward so as to get nearer to the face.

Each of said intermediate section has a dimension in a range of 2 to 10 mm as viewed in the vertical direction.

Each of the lenses is coated with pigment or dye in order to lessen the see-through clarity of the intermediate section.

Light blocking or light scattering tape is installed on each of the lenses in order to lessen the see-through clarity of the intermediate section.

Each of the lenses is formed on its surface in the intermediate section with a plurality of fine irregularities in order to lessen the see-through clarity of the intermediate section.

The lens assemblies are provided therearound with eye-cups, respectively, belt-like portions formed integrally with the eye-cups extend across the lens assemblies between inner and outer ends of the lens assemblies in close contact with outer surfaces of the lens assemblies so as to cover the intermediate sections of respective the lens assemblies.

The term “see-through clarity” used herein refers to an easiness with which a target can be visually recognized through lens and can be specified by total luminous transmittance and/or parallel luminous transmittance. In the present invention, the see-through clarity is described to become lower as the luminous transmittance decreases.

### BRIEF DESCRIPTION OF THE DRAWINGS

FIG. 1 is a perspective view of swimming goggles according to one embodiment of the invention;

FIG. 2 is a sectional view taken along the line II-II in FIG. 1;

FIG. 3 is a perspective view of swimming goggles according to an alternative embodiment of the invention; and

FIG. 4 is a sectional view taken along the line IV-IV in FIG. 3.

### DETAILED DESCRIPTION OF THE PREFERRED EMBODIMENTS

Details of swimming goggles according to the present invention will be more fully understood from the description given hereunder with reference to the accompanying drawings.

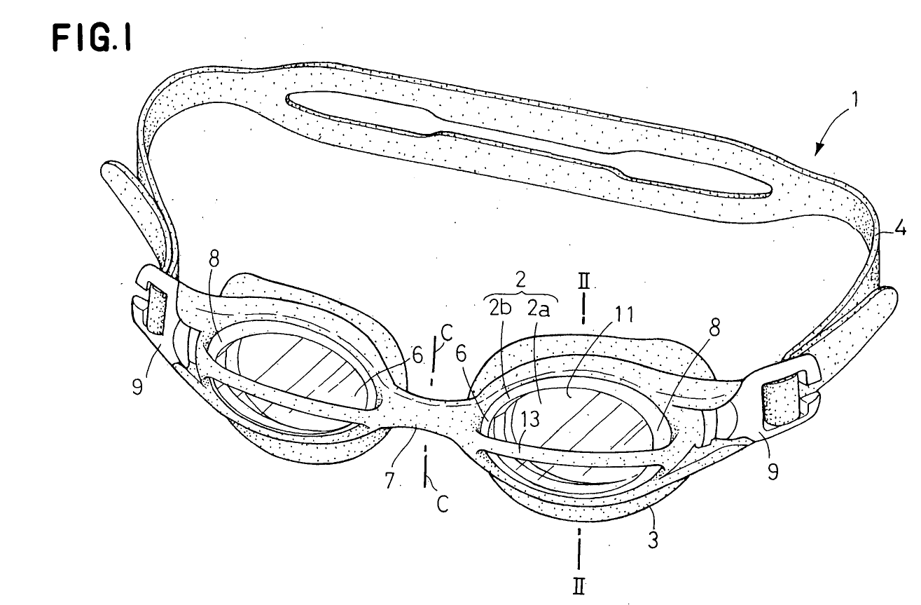

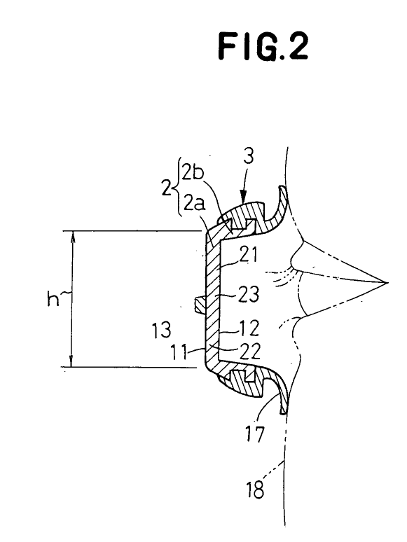

FIG. 1 is a perspective view of swimming goggles 1. The swimming goggles 1 has a horizontal direction extending transversely of the wearer's head and a vertical direction which is orthogonal to the horizontal direction. The swimming goggles 1 are symmetrically configured about a center line C-C extending in the vertical direction and comprise a pair of lens assemblies 2, a pair of eye-cups 3 and a strap 4 adapted to be put around the wearer's head to fasten the goggles 1 thereon. In a vicinity of inner ends 6 of the respective lens assemblies 2, the eye-cups 3 are connected with each other by a link 7 serving as a nose belt. Each of the lens assemblies 2 comprises a lens 2a and a peripheral frame 2b wherein the lens 2a is made of see-through material and the peripheral frame 2b is provided with the eye-cup 3 attached thereto. In a vicinity of outer ends 8 of the respective lens assemblies 2, the strap 4 is length-adjustably connected to the respective eye cups 3 by means of buckles 9. Each of the eye-cups 3 includes a belt-like portion 13 extending across the lens 2a in close contact with an outer surface 11 (See FIG. 2) of the lens assembly 2 between the inner and outer ends 6, 8.

FIG. 2 is a sectional view taken along the line II-II in FIG. 1. Along this line II-II, the lens 2a has its vertical maximum dimension. The lens assembly 2 has the outer surface 11 and an inner surface opposed to the wearer's face indicated by imaginary lines. The eye-cup 3 is detachably or undetectably attached to the peripheral frame 2b of the lens assembly 2. A skirt 17 of the eye-cup 3 is adapted to be held in close contact with the wearer's face 18. The belt-like portion 13 of the eye-cup 3 is laid, more preferably fastened in close contact with the outer surface 11 of the lens 2a so as to divide the lens 2a, as viewed in the vertical direction of the lens 2a, an upper section 21 lying above the belt-like portion 13, a lower section 22 lying below the belt-like portion 13 and an intermediate section 23 covered with the belt-like portion 13. While vertical dimensions (widths) of these sections 21, 22, 23 may be appropriately selected, the belt-like portion 13 preferably has a width in a range of 2 to 10 mm. Preferably, the belt-like portion 13 is positioned to bisect a vertical dimension h of the lens assembly or position slightly aside downward so that a visual field through the upper section 21 may be larger than a visual field through the lower section 22.

The lens assembly 2 is made of clear and colorless or clear and colored hard materials while the eye-cup 3 including the belt-like portion 13 is made of clear and colored or colored and opaque flexible elastic materials. To make the eye-cup 3 including the belt-like portion 13 substantially undetachable from the lens assembly 2, the eye-cup 3 may be attached to the lens assembly 2 under elastic deformation or using adhesives, or the eye-cup 3 may be injection molded using a lens assembly inserted mold. In this way, the belt-like portion 13 can be welded to the outer surface 11 of the lens assembly 2.

When the wearer of the swimming goggles 1 having such lens assemblies 2 intends to look at any target through the lenses 2a, his or her visual line is obstructed by the belt-like portions 13 and necessarily directed to the upper section 21 or the lower section 22. Particularly during competitive swimming or exercise for competitive swimming, the visual line of the wearer is necessarily directed to the upper section 21 and the wearer looks forward horizontally through the upper section 21. Thus, substantially no extra sight gets into the wearer and the wearer's concentration is sharpened. During walking, for example, after the competitive swimming, the wearer can selectively use the upper section 21 and the lower section 22. In other words, these swimming goggles 1 provide a relatively wide visual field so far as both the upper section 21 and the lower section 22 are used, but allow the visual field thereof to be reduced substantially to the upper section 21 alone during the competitive swimming.

The belt-like portion 13 in the swimming goggles 1 is provided to make a see-through clarity of the intermediate section 23 of the lens 2a lower than a see-through clarity of the upper and lower sections 21, 22 of the lens 2a. Such belt-like portion 13 may be of any appropriate color and any appropriate shade. Alternatively, the belt-like portion 13 made of the same materials as the eye-cup 3 may be replaced by colored adhesive tape having the same width as the belt-like portion 13 installed on the outer surface 11 or the inner surface 12 in order to lessen the see-through clarity of the intermediate section 23. It is also possible to partially coat the outer surface 11 or the inner surface 12 with pigment or dye. Furthermore, it is also possible to form the outer surface 11 or the inner surface 12 with a plurality of irregularities to obtain the stain finished light scattering surface 11 or 12 and thereby to lessen the see-through clarity of the intermediate section 23. In an extreme case, the intermediate section 23 may have total luminous transmittance and/or parallel luminous transmittance of 0%, i.e., may present no see-through clarity.

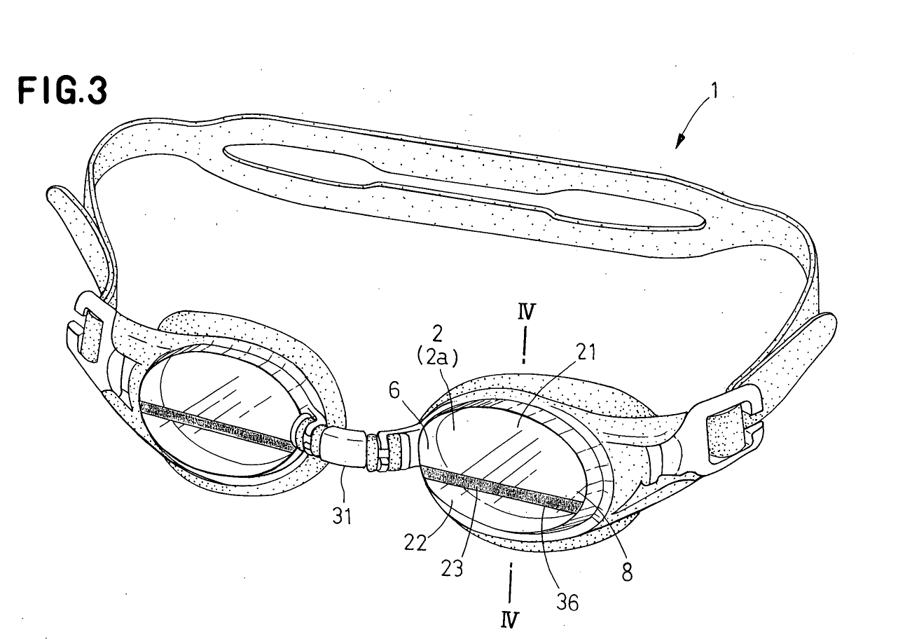

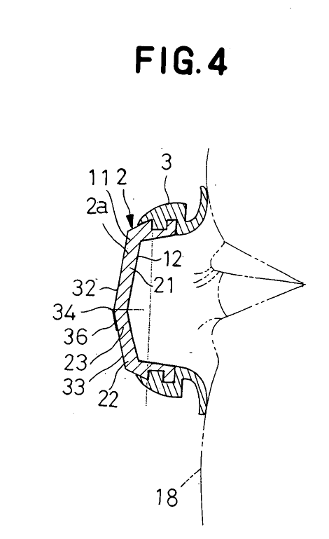

FIG. 3 is a view similar to FIG. 1 of swimming goggles according to an alternative embodiment of the invention and FIG. 4 is a sectional view taken along the line IV-IV in FIG. 3. The swimming goggles 1 according to this embodiment is distinguished from the swimming goggles shown by FIG. 1 in that a pair of lens assemblies 2 are connected to each other by means of a nose belt 31 provided separately of the eye cups 3. Each of the lenses 2a in these swimming goggles 1 comprises, as will be apparent from FIG. 4, an upper section 32 obliquely extending upward down toward so as to get nearer to the wearer's face 18 and a lower section 33 extending downward so as to get nearer to the wearer's face 18 so that these upper and lower sections 32, 33 cross each other in a vicinity of a vertically middle level or at a level slightly lower than the middle level so as to form an edge 34 extending from the inner surface toward the outer surface 11. Immediately below the edge 34, light blocking or light scattering adhesive tape 36 is applied to the outer surface 11 so as to extend along the edge 34. The tape 36 has a width in a range of 2 to 10 mm and extends across the lens 2a between the inner end 6 and the outer end 8. The tape 36 divides the lens 2a, in the vertical direction, into the upper section 21, the lower section 22 and the intermediate section 23. The intermediate section 23 covered with the tape 36 has its total luminous transmittance and/or parallel luminous transmittance lower than those of the upper and lower sections 21, 22 and presents the see-through clarity correspondingly lower than those of the upper and lower sections 21, 22.

Similarly to the swimming goggles shown by FIGS. 1 and 2, the swimming goggles shown in FIGS. 3 and 4 are formed with the intermediate sections 23 on the respective lenses 2 which present the relatively low see-through clarity. Therefore, the swimming goggles according to this embodiment also necessarily directs the visual line of the wearer to the upper section 21 particularly during competitive swimming. The upper section 21 obliquely extending upward and the lower section 22 obliquely extending downward are effective to enlarge the visual field.

The swimming goggles according to the present invention are primarily characterized in that each of the lenses is divided, in the vertical direction, into the upper section, the lower section and the intermediate section interposed between these two sections wherein the intermediate section has a see-through clarity lower than that of the upper and lower sections. While the total visual field of the lenses is relatively wide, the visual line of the wearer is necessarily directed to the upper section during competitive swimming and thereby facilitates the wearer to look at forward in the horizontal direction. In addition, any sight unnecessary for competitive swimming rarely comes into sight and the wearer can easily sharpen his or her concentration.

What is claimed is:

1. Swimming goggles comprising:
a pair of lens assemblies;
a link connecting said lens assemblies to each other;
a strap put around a head of a swimming goggles wearer; and
said lens assemblies having a transverse direction horizontally extending across said head and a longitudinal direction vertically extending orthogonally to said transverse direction and the lens included in each of said lens assemblies being divided into an intermediate section extending across the lens at a substantially middle level as viewed in a vertical direction, an upper section lying above the intermediate section and a lower section lying below the intermediate section wherein the intermediate section presents a see-through clarity lower than those presented by the upper section and the lower section.

2. The swimming goggles according to claim 1, wherein said see-through clarity is specified by total luminous transmittance or parallel luminous transmittance of said intermediate sections.

3. The swimming goggles according to claim 2, wherein said total luminous transmittance or said parallel luminous transmittance of said intermediate sections is substantially 0%.

4. The swimming goggles according to claim 1, wherein each of said lenses has an inner surface opposed to a swimming goggles wearer's face and an outer surface facing away from said wearer's face and wherein each of said upper section obliquely extends upward so as to get nearer to said face and each of said lower section obliquely extends downward so as to get nearer to said face.

5. The swimming goggles according to claim 1, wherein each of said intermediate section has a dimension in a range of 2 to 10 mm as viewed in said vertical direction.

6. The swimming goggles according to claim 1, wherein each of said lenses is coated with pigment or dye in order to lessen said see-through clarity of said intermediate section.

7. The swimming goggles according to claim 1, wherein light blocking or light scattering tape is installed on each of said lenses in order to lessen said see-through clarity of said intermediate section.

8. The swimming goggles according to claim 1, wherein each of said lenses is formed on its surface in said intermediate section with a plurality of fine irregularities in order to lessen said see-through clarity of said intermediate section.

9. The swimming goggles according to claim 1, wherein said lens assemblies are provided therearound with eye-cups, respectively, belt-like portions formed integrally with said eye-cups extend across said lens assemblies between inner and outer ends of said lens assemblies in close contact with outer surfaces of said lens assemblies so as to cover said intermediate sections of respective said lens assemblies.
